(19) United States
(12) Patent Application Publication
(10) Pub. No.: US 2023/0075468 A1
(43) Pub. Date: Mar. 9, 2023

(54) TITANIUM DIOXIDE COATED SUBSTRATE WITH ADHESIVE

(71) Applicant: Deloren E. Anderson, Crosby, MN (US)

(72) Inventor: Deloren E. Anderson, Crosby, MN (US)

(21) Appl. No.: 17/929,982

(22) Filed: Sep. 6, 2022

Related U.S. Application Data
(60) Provisional application No. 63/260,914, filed on Sep. 3, 2021.

(51) Int. Cl. B01J 23/50; B01J 35/00; C09J 7/29; A61L 2/08; A61L 2/26

(52) U.S. Cl. CPC B01J 23/50; B01J 35/004; C09J 7/29; A61L 2/088; A61L 2/26; C09J 2301/41

(57) ABSTRACT

A device includes a flexible substrate having a length and width greater than a depth of the substrate. A photocatalytic titanium dioxide or silver photocatalytic titanium dioxide coating is supported by the substrate on a first surface defined by the length and width of the substrate. An adhesive is disposed on a second surface of the substrate opposite the first surface of the substrate.

### RELATED APPLICATION

This application claims priority to U.S. Provisional Application Ser. No. 63/260,914 (entitled TITANIUM DIOXIDE COATED SUBSTRATE WITH ADHESIVE, filed Sep. 3, 2021) which is incorporated herein by reference.

### BACKGROUND

Humans touch many surfaces during an ordinary day. Some of the surfaces, such door handles and shopping cart handles are touched by many different people and can carry harmful bacteria, viruses, and other organic microbes that can be spread, potentially infecting many people. Washing such surfaces can be time consuming, logistically difficult, and if not well done, ineffective in preventing the spread of illnesses.

### SUMMARY

A device includes a flexible substrate having a length and width greater than a depth of the substrate. A photocatalytic titanium dioxide or silver photocatalytic titanium dioxide coating is supported by the substrate on a first surface defined by the length and width of the substrate. An adhesive is disposed on a second surface of the substrate opposite the first surface of the substrate.

### BRIEF DESCRIPTION OF THE DRAWINGS

FIG. 1 is a perspective view of a coated substrate with adhesive.

FIG. 2 is cross section view of a portion of the coated substrate of FIG. 1.

FIGS. 3A, 3B, and 3C are graphs illustrating electron-hole recombination rates for different solutions.

FIG. 4 is a perspective view of a coated substrate adhered to a surface frequently touched by humans.

FIG. 5 is a perspective view of a coated substrate adhered to the handle of a shopping cart.

FIG. 6 is a perspective view of a coated substrate adhered to the handle of a shopping basket.

FIG. 7 is a perspective view of a coated substrate adhered to a door handle.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

In some embodiments Titanium Dioxide or Silver Titanium Dioxide is added to a substrate to provide multiple characteristics. Titanium Dioxide or Silver Titanium Dioxide is photocatalytic such that it is activated by light. The substrate, commonly referred to as a backing, may be a flexible material such as commonly used for tape or stickers, and includes an adhesive to be applied to one or more surfaces that are commonly touched by humans. Typical backings or carriers may be made of paper, plastic film, cloth, foam, foil, or other suitable material. The Titanium Dioxide or Silver Titanium Dioxide may be added to the substrate during formation of the substrate and may also be added to an outside of the substrate following substrate formation into a desired shape such as a sticker or elongated tape of varying widths adapted for various particular applications, such as adhering to a door handle, shopping cart, basket handle, or other commonly touched surface. The elongated tape may be wound into a roll of tape in some embodiments for use in applying to a handle or other commonly touched structure.

Titanium Dioxide or Silver Titanium Dioxide may operate as a catalyst on the substrate and help protect multiple people that may touch the substrate.

In portions of the side of the substrate that is coated and exposed to the emitted visible light in the 400 nm range or higher, the Titanium Dioxide or Silver Titanium Dioxide acts as a photo catalyst for degradation of organic molecule pollutants, and microbes with or without light. In one embodiment, the Titanium Dioxide or Silver Titanium Dioxide may be in the form of nanoparticles or crystals which may be used to coat the substrate. The nanoparticles may be formed by extracting TiO2 or Ag TiO2 from peroxides and heating the particles to 250-260° C. The use of crystalline particles, including nanometer sized crystalline particles may both increase the surface area and hence photocatalytic efficiency of the Titanium Dioxide or silver (Ag) Titanium oxide and enable activation with visible light. In one embodiment, the crystalline particles are on average, less than 20 nm in diameter.

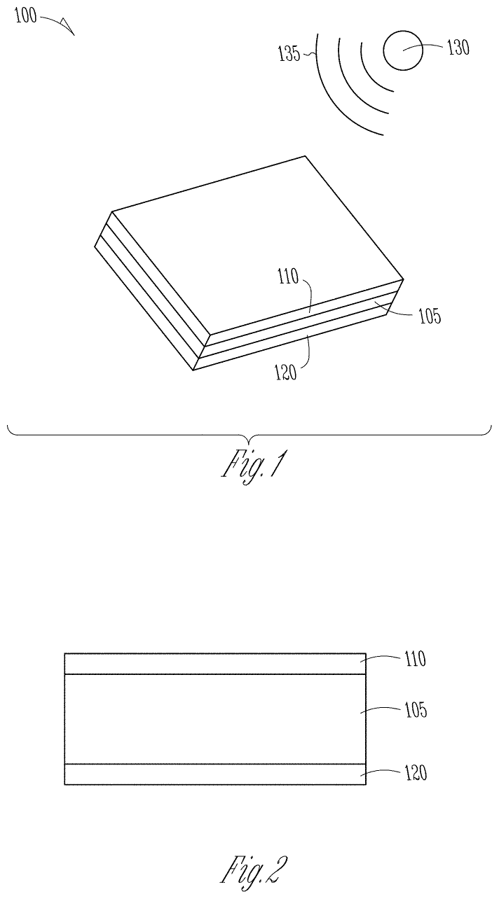

FIG. 1 is a perspective view of a device 100 including a substrate 105 having silver photocatalytic titanium dioxide coating 110 supported by the substrate 100. FIG. 2 is a cross section view of a portion of the substrate 100. Substrate 100 is formed of a material such as that used for tape or stickers. Masking tape is one example substrate. that provides acts as an air filter. In one embodiment, the coating 110 contains photocatalytic titanium dioxide or silver photocatalytic titanium dioxide, optionally in crystalline form, that covers at least one side of the substrate 100. Substrate 100 also includes an adhesive 120 to facilitate attachment of the substrate with coating to desired surfaces, such as door or cart handles.

Many different light sources, such as that represented at 130 may emit light 135 to activate the coating 110. The light source 130 may comprise natural sunlight, LED light, or other light which may include visible and/or UV light to activate the coating.

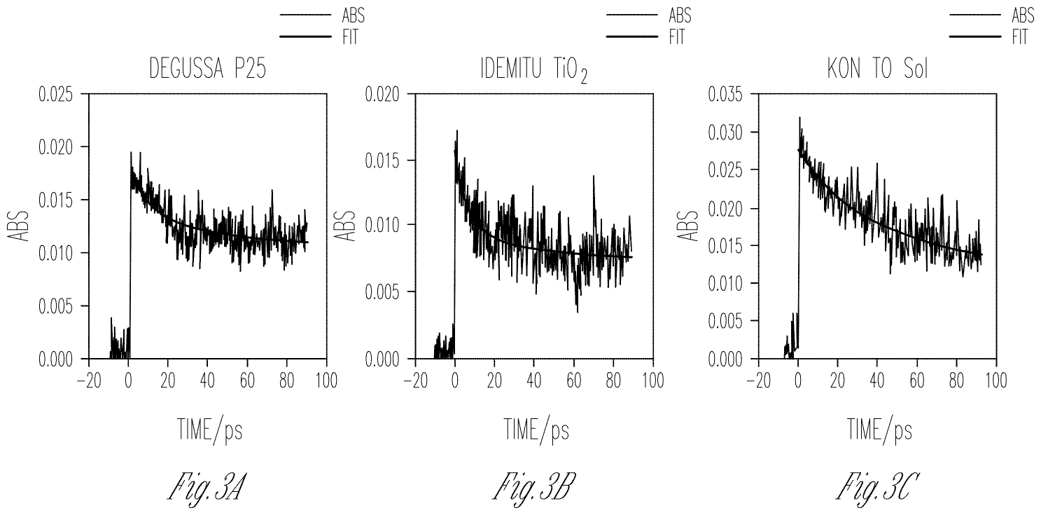

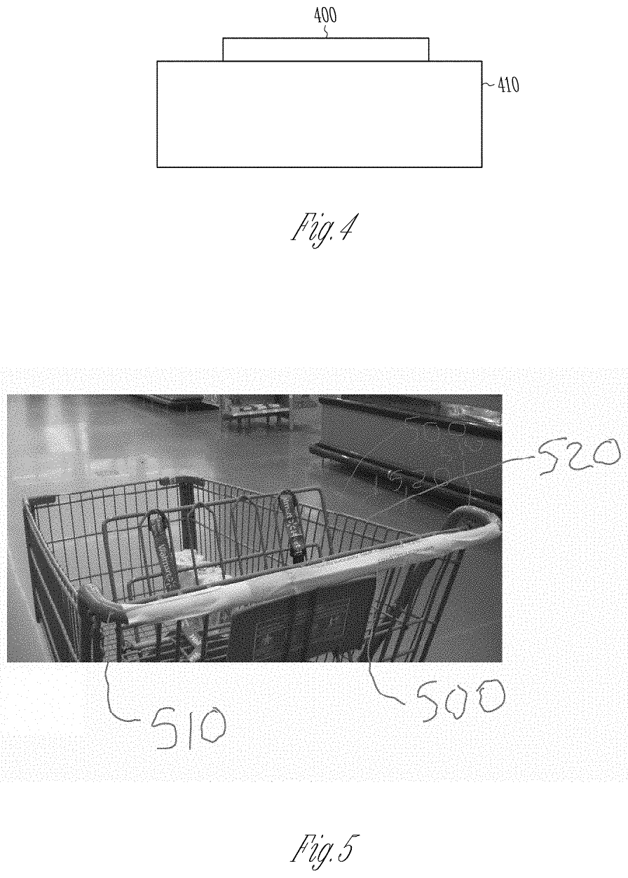

FIGS. 3A, 3B, and 3C are graphs illustrating TiO₂ electron-hole recombination dynamics for different solutions.

One example method of producing the Titanium Dioxide or Silver Titanium Dioxide includes:

A 30% solution of hydrogen peroxide (20 ml) was added to and stirred with a solution (500 ml) of a 60% aqueous solution of titanium tetrachloride (5 ml) diluted with distilled water to prepare a transparent, brown solution. Ammonia water (1:9) was added dropwise to the solution to regulate the pH of the solution to 7, thereby preparing a transparent, yellow solution. The obtained solution was let stand at 25 degrees C. for a whole day and night to obtain yellow precipitates.

Distilled water was added to the precipitates after filtered and washed to prepare a solution (about 150 ml), and a cation exchange resin and an anion exchange resin, each in an amount of 25 g, were charged into the solution, which was then let stand for 30 minutes for removal of cationic and anionic substances.

An H⁺ substituted type resin obtained by treating Amberite IR120B (Na⁺ substituted type, and made by Organo Co., Ltd.) with 2N hydrochloric acid for 1 hour was used for the cation ion exchange resin, and an OH⁻ substituted type resin obtained by treating Amberite IRA410 (Cl⁻ substituted type, and made by Organo Co., Ltd.) with IN sodium hydroxide for 1 hour was used for the anion exchange resin.

Powders obtained by drying the resultant yellow precipitates at 25° C. were measured with an X-ray diffactometer (RAD-B made by Rigaku Denki Co., Ltd.) using a copper target while it was operated at an acceleration voltage of 30 kV and with a current of 15 mA. The obtained precipitates were found to be in an amorphous state.

On the other hand, the powders obtained by drying at 25° C. were mixed with potassium bromide to prepare a tablet. According to the potassium bromide tablet method, the tablet was then measured using a Fourier transform infrared absorption spectrometer (FT/IR-5300 made by Nippon Bunko Co., Ltd.) in combination with a transmission technique. Absorption was found in the vicinity of 900 cm⁻¹, indicating the presence of peroxo groups.

Then, the ion exchange resins were removed by filtration, and distilled water was added to prepare a solution (about 180 ml), which was in turn cooled with ice water. Thereafter, a 30% solution of hydrogen peroxide (20 ml) was added to the solution, followed by cooling. After the lapse of 1 hour, a transparent, yellow solution (200 ml) containing titanium was obtained.

After a one-month or longer storage in a refrigerator at 7° C., the solution remained unchanged. Five days after preparation, the pH of the transparent, yellow solution was 5.1. Powders obtained by drying this solution at normal temperature, too, were similarly measured by X-ray diffraction. From the results of X-ray diffraction, it was found that the powders were in a non-crystalline state having no peak indicative of crystallinity. Results of a Fourier transform infrared spectroscopy resulted in absorption being found in the vicinity of 900 cm⁻¹, indicating the presence of a number of peroxo groups.

In one embodiment, the substrate material is flexible plastic, vinyl, or tightly woven cloth.

Optical coupling of the coating to light causes the material coated or infused with Titanium oxide TiO2 to act as a photo-catalyst producing a bacteria stat, fungus stat, virus stat, etc., that removes organic molecules and microbes. The silver particles may be added and operate similarly without the need for exposure to light.

The coating may be applied with an air brush in one embodiment or applied by soaking or submerging the substrate in the solution.

FIG. 4 is a perspective view of a coated substrate 400 adhered to a surface 410 frequently touched by humans.

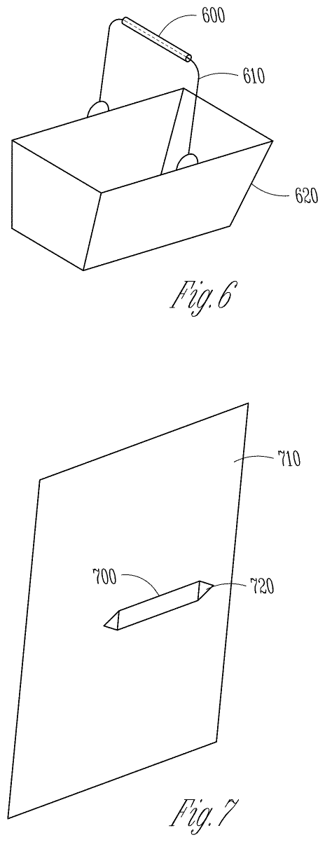

FIG. 5 is a perspective view of a coated substrate 500 adhered to the handle 510 of a shopping cart 520. The substrate 500 may be a tape that has a width sufficient to wrap one or more times around the handle 500 with a length that may be tripped to the length of the handle, or may be wrapped in a spiral manner if the width of the tape is not wide enough.

FIG. 6 is a perspective view of a coated substrate 600 adhered to the handle 610 of a shopping basket 620.

FIG. 7 is a perspective view of a coated substrate 700 adhered to a door 710 handle 720.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A device comprising:
a flexible substrate having a length and width greater than a depth of the substrate;
a photocatalytic titanium dioxide coating supported by the substrate on a first surface defined by the length and width of the substrate; and
an adhesive disposed on a second surface of the substrate opposite the first surface of the substrate.

2. The device of claim 1 wherein the substrate is formed as a sticker.

3. The device of claim 1 wherein the substrate is formed as a tape.

4. The device of claim 3 wherein the tape comprises roll of tape.

5. The device of claim 1 wherein the coating comprises crystallized Silver doped Titanium Dioxide.

6. The device of claim 1 wherein the coating is applied as a suspension to a first surface of the material.

7. The device of claim 1 wherein the coated first surface is configured to be exposed in response to the substrate being adhered to a handle.

8. The device of claim 1 where in the photocatalytic Titanium Dioxide is photocatalytic at at least 400 nm wavelength light.

9. The device of claim 1 where in the photocatalytic Titanium Dioxide is photocatalytic at at least 500 nm wavelength light.

10. The device of claim 1 where in the photocatalytic Titanium Dioxide is photocatalytic at human visible wavelength light.

11. The device of claim 1 wherein exposing the coating to light activates the photocatalytic titanium dioxide layer to break down organic particles, and microbes.

12. The device of claim 11 wherein the coating comprises crystallized Silver doped Titanium Dioxide.

13. A device comprising:
a flexible substrate having a length and width greater than a depth of the substrate;
a photocatalytic silver titanium dioxide coating supported by the substrate on a first surface defined by the length and width of the substrate; and
an adhesive disposed on a second surface of the substrate opposite the first surface of the substrate.

14. The device of claim 13 wherein the substrate is formed as a sticker.

15. The device of claim 13 wherein the substrate is formed as a tape.

16. The device of claim 15 wherein the tape comprises roll of tape.

17. The device of claim 13 wherein the coating comprises crystallized Silver doped Titanium Dioxide.

18. The device of claim 13 wherein the coating is applied as a suspension to a first surface of the material.

19. The device of claim 13 wherein the coated first surface is configured to be exposed in response to the substrate being adhered to a handle.

20. The device of claim 13 where in the photocatalytic Titanium Dioxide is photocatalytic at human visible wavelength light and wherein exposing the coating to light activates the photocatalytic titanium dioxide layer to break down organic particles, and microbes.
